(19) United States
(12) Patent Application Publication
(10) Pub. No.: US 2024/0390181 A1
(43) Pub. Date: Nov. 28, 2024

(54) Electrically Operable, Multi-Layer Electric Blanket for Patients

(71) Applicant: GENTHERM MEDICAL LLC, Cincinnati, OH (US)

(72) Inventors: Michael Weiss, Benediktbeuem (DE); Florian Sandmair, Ried (DE)

(21) Appl. No.: 18/712,455

(22) PCT Filed: Dec. 1, 2022
(86) PCT No.: PCT/DE2022/000116

§ 371 Date: May 22, 2024

(30) Foreign Application Priority Data
Dec. 2, 2021 (DE) 10 2021 005 983.2

(51) Int. Cl. A61F 7/00; A47G 9/02; B32B 5/02; B32B 5/26; H05B 1/02; H05B 3/34

(52) U.S. Cl. CPC A61F 7/0097; A47G 9/0215; A47G 9/0223; A61F 7/007; B32B 5/022; B32B 5/266; H05B 1/0272; H05B 3/342; B32B 2262/0284; B32B 2307/202; B32B 2307/304; B32B 2535/00; H05B 2203/003

(57) ABSTRACT

A multi-layer electrically operable patient electric blanket that includes a heating layer and a textile layer connected to the heating layer in a planar manner, the heating layer has one or a plurality of electrically conductive and non-destructively deformable heating tracks.

### CROSS REFERENCE TO RELATED APPLICATIONS

This application is a US National Stage of entry of PCT/DE2022/000116 filed on Dec. 1, 2022, which claims priority to DE 10 2021 005 983.2 filed on Dec. 2, 2021, all of which are hereby incorporated by reference herein for all purposes.

### FIELD

The invention relates to an electrically operable, multi-layer patient electric blanket having a heating layer that has one or a plurality of electrically conductive and non-destructively deformable heating tracks.

### BACKGROUND

Patient electric blankets are employed in particular for treating patients with heat outside of in-patient medical facilities. Patient electric blankets may thus be used, for example, for providing heat outdoors or during transport to an in-patient medical facility.

Because patient electric blankets are increasingly designed as single use or disposable articles, there are always efforts to be able to produce these patient electric blankets as cost-effectively as possible. When producing such patient electric blankets, it must be taken into account that the electrically conductive heating tracks must be insulated and protected against environmental factors so that the patient does not come into contact with the heating tracks and the patient electric blanket has a certain mechanical resilience.

In the patient electric blankets employed in the prior art, the heating tracks used to date are covered and insulated by a PET cover film. Such PET cover films are comparatively expensive, however. In addition, a comparatively large quantity of adhesive is required for affixing such PET cover films to the heating layer, so that the materials and production costs are high. In addition, the use of PET cover films leads to a stiff and not particularly bendable electric blanket structure, the shape of which does not adequately adapt to the body of the patient.

To improve the shape adapting properties of such patient electric blankets, it has become common to provide one or a plurality of layers of the patient electric blanket with slits so that its flexibility is enhanced. However, the improvement in the deformation properties of a patient electric blanket provided by adding deformation slits is not adequate for a number of applications. Furthermore, the comfort of the known patient electric blankets is also significantly adversely affected by an unpleasant crackling sound created when the blanket is deformed.

### SUMMARY

The underlying object of the invention is thus to increase the ability of a patient electric blanket to adapt in shape to the body of the patient and/or to enhance the comfort of a patient electric blanket, wherein at the same time the production costs are to be reduced.

This object is attained using a patient electric blanket of the type cited in the foregoing, wherein the inventive patient electric blanket comprises a textile base layer connected in a planar manner to the heating layer.

A textile base layer can be produced and applied to the heating layer at significantly less cost than a PET cover layer. The joining of heating layer and textile base layer in a planar manner is possible at comparatively low cost and time required. The textile base layer leads to the patient electric blanket having a low degree of stiffness and thus being able to adapt optimally to the body shape of a patient. Adding slits to the material layers of the patient electric blanket, which is time-consuming and expensive, is not necessary. Furthermore, an unpleasant crackling sound is avoided, or, if the latter is caused by other material layers present in the patient electric blanket, is significantly reduced due to the noise-damping effect of the textile base layer. The multi-layer patient electric blanket is preferably embodied as a single-use or disposable article.

In one preferred embodiment of the inventive patient electric blanket, the textile base layer is embodied from a non-woven fabric. Since the non-woven fabric is a thermally insulating material, the thermal insulation properties of the patient electric blanket is further increased by the use of a textile base layer embodied from non-woven fabric. Moreover, the non-woven fabric ensures uniform temperature distribution, so that the patient can be provided uniform heat. The textile base layer is in particular embodied from a polyethylene terephthalate (PET) non-woven fabric. The PET non-woven fabric comprises at least 80% polyester fibers, preferably at least 90% polyester fibers. Alternatively or in addition, the PET non-woven fabric comprises at least at least 10% Co-PET thermoplastic fibers that stabilize the non-woven fabric after needling using a thermal process. The mass per unit area of the non-woven fabric is preferably between 80 g/m² and 120 g/m². The mass per unit area of the non-woven fabric is preferably about 100 g/m², for example.

In one further preferred embodiment of the inventive patient electric blanket, the base layer is arranged immediately on top of the heating layer, in particular on the one or on the plurality of heating tracks of the heating layer. The heating layer can comprise a plurality of films, wherein the base layer can be arranged immediately on top of one, a plurality of, or all of the films of the heating layer. The base layer is also arranged immediately on top of the heating layer when there is a joining material disposed between the base layer and the heating layer, which joining material connects the base layer and the heating layer to one another in a material fit and in a planar manner. The joining material can be an adhesive, for example. There is preferably no separate insulating layer, for example an insulating PET cover layer, between the base layer and the heating layer.

Moreover, an inventive patient electric blanket is advantageous in which the textile base layer and the heating layer are bonded to one another. The textile base layer and the heating layer are preferably bonded to one another by means of a hot melt adhesive. By using a textile base layer, the bonding process can take place at low bonding or laminating temperatures, so that the bonding process is simplified and accelerated. Due to the low bonding or laminating temperatures, the joining process for the textile base layer and the heating layer also occurs with a low use of energy. Due to the use of a textile base layer, fewer adhesive folds and uneven areas occur during the bonding process, as well, so that the patient electric blanket has a uniform patient contact surface with a high degree of contact comfort.

In one further preferred embodiment, the inventive patient electric blanket has one or a plurality of temperature sensors, wherein the one or the plurality of temperature sensors are each arranged in a temperature detection region of the patient electric blanket. The one or the plurality of temperature sensors detect the temperature in the respective temperature detection region. The one or the plurality of temperature sensors can each be embodied as thermistors, in particular as negative temperature coefficient thermistors or positive temperature coefficient thermistors. The one or the plurality of temperatures sensors are preferably designed to be operated with a supply voltage of less than 30 volts, in particular less than 26 volts, particularly preferably with a supply voltage of about 22 volts. The textile base layer is sufficiently insulating for such thermistors. The one or the plurality of temperature sensors can be surface-mounted temperature sensors. For example, the one or the plurality of temperature sensors are cast, so that they can be protected from mechanical stress by the casting material. The casting material can be an adhesive, for example.

In one refinement of the inventive patient electric blanket, the one or the plurality of temperature sensors are each covered by a cover material, in particular a textile cover material. For example, the base layer has a recess in the region of the one or each of the plurality of temperature sensors, so that after the heating layer and the base layer have been connected the one or the plurality of temperature sensors can be placed into the recesses of the base layer. Once the one or the plurality of temperature sensors has been placed into and fixed in the respective recesses, the one or the plurality of temperature sensors must still be covered. The cover material used for this purpose can be bonded to the region of the one or the plurality of temperature sensors in the form of affixable patches, for example. The cover material is preferably the same as the material from which the base layer is also embodied.

The inventive patient electric blanket is furthermore advantageously refined by a connection region, wherein the connection region can have one or a plurality of electrical power supply connectors and/or one or a plurality of sensor connectors. Electrical energy from an energy source external to the blanket can be provided to the one or the plurality of heating tracks via the one or the plurality of electrical power supply connectors. The one or the plurality of sensor connectors are connected to the one or the plurality of temperature sensors, wherein an external evaluation device for temperature detection can be connected to the one or the plurality of sensor connectors. The energy source and the evaluation device can be components of a control module that can be connected to the patent electric blanket and is external thereto. The one or the plurality of electrical supply connectors and the one or the plurality of sensor connectors can be plug-in connectors that can be connected to the control module via one or a plurality of plugs. The control module can be designed, for example, to control the operation of the patient electric blanket. The control module is preferably designed to regulate the temperature of the patient electric blanket, taking into account the temperature values detected via the one or the plurality of temperature sensors. The control module can be designed for multiple usages, so that the control module can be connected to different patient electric blankets embodied as single-use articles. During medical usage, the electric blanket is prevented from becoming too hot and the electric blanket not heating are to be prevented. To counter it becoming too hot, the patient electric blanket has a plurality of temperature sensors, for example eight temperature sensors, that are distributed, in particular distributed evenly, over the surface area of the patient electric blanket. Effective protection against overheating, even for normal folding of the patient electric blanket, is provided due to the arrangement of the temperature sensors. Temperature regulation is preferably based on the temperature value for the hottest temperature sensor, so that it is not even possible for a segment of the patient electric blanket to overheat, for example if the latter is folded.

Moreover, an inventive patient electric blanket is advantageous in which the heating layer includes one or a plurality of heating films carrying the heating tracks. The one or the plurality of heating films can be plastic films onto which the one or the plurality of heating tracks are applied or into which the one or the plurality of heating tracks are integrated. If the heating layer includes a plurality of heating films, the heating films can be arranged adjacent to one another, overlapping offset laterally to one another, and/or above one another. The heating layer can thus comprise one film ply or a plurality of film plies.

In one further preferred embodiment of the inventive patient electric blanket, the power supply connectors are components of the one or the plurality of heating films. The heating films preferably extend into the connection region of the patient electric blanket. In the connection region, the heating tracks can be connected to electrically conductive contacting bodies that facilitate the connection of an energy source external to the blanket.

Moreover, an inventive patient electric blanket is preferred in which the one or the plurality of sensor connectors are components of a contacting film, wherein the contacting film includes one or a plurality of contacting tracks connected to the one or the plurality of temperature sensors. The contacting film can be a component of the heating layer. The heating layer preferably includes at least one contacting film and one or a plurality of heating films carrying the heating tracks. The contacting film and the heating films can be arranged overlapping one another, at least in segments. The contacting film and the heating films can be bonded to the textile base layer.

In one further preferred embodiment of the inventive patient electric blanket, the one or the plurality of heating tracks run in a meandering pattern within a heating region, wherein the heating region extends across at least 70% of the patient electric blanket, preferably across at least 80% of the patient electric blanket. Using a meandering pattern for the one or the plurality of heating tracks can be used to generate heat over a large surface area, wherein the temperature distribution is comparatively uniform due to the meandering pattern. The heating tracks preferably include a plurality of track segments running parallel to one another.

In one further preferred embodiment, the inventive patient electric blanket has a plurality of heating fields, wherein each heating field includes one or a plurality of heating tracks. The heating fields are preferably wired in series. Thus, if one heating track is damaged, all of the heating fields become inoperative. This is detected via the temperature sensors, so that an alarm or warning can be output. In this way, medical personnel can react promptly to the failure of the patient electric blanket. The series wiring of the heating fields prevents a sub-region of the patient electric blanket from remaining cold unnoticed when there is a malfunction of a heating field and leading to a negative result for the patient, in particular to undercooling.

In one refinement, the inventive patient electric blanket has a first textile thermal insulation layer that is connected in a planar manner to the heating layer on the side of the heating layer facing away from the base layer. Alternatively or in addition, the inventive patient electric blanket has a second textile thermal insulation layer that is connected in a planar manner to the base layer on the side of the base layer facing away from the heating layer. The textile thermal insulation layers ensure thermal insulation and uniform heat distribution. The thermal insulation layers can be glued or laminated on.

In one preferred embodiment of the patient electric blanket, the first textile thermal insulation layer and/or the second textile thermal insulation layer each include one or a plurality of non-woven fabric plies, in particular two non-woven fabric plies. The non-woven fabric plies of the thermal insulation layers can include, for example, polyethylene terephthalate non-woven fabric plies or can be embodied as polyethylene terephthalate non-woven fabric plies. The plurality of non-woven fabric plies can be bonded to one another.

Moreover, an inventive patient electric blanket is preferred that has a first textile comfort layer that forms a first outer side of the patient electric blanket. Alternatively or in addition, the patient electric blanket can have a second textile comfort layer that forms a second outer side of the patient electric blanket. The first textile comfort layer can be applied, in particular bonded, to the outside of the first textile thermal insulation layer. The second textile comfort layer can be applied, in particular bonded to the outside of the second textile thermal insulation layer. The textile comfort layers can come into contact with a patient or user of the patient electric blanket, even into contact with the skin. The textile comfort layers are thus designed to be brought into contact with a patient. The outside of each of the textile comfort layers has a patient contact surface. The patient contact surfaces are preferably non-woven fabric surfaces. Such a non-woven fabric surface is comparatively soft, flexible, quiet, noiseless, and in addition sound-absorbent. The textile comfort layers embodied from non-woven fabric adapt particularly well to the body shape of a patient.

The inventive patient electric blanket is furthermore advantageously refined in that the first textile comfort layer and/or the second textile comfort layer each include at least one non-woven fabric ply, in particular a non-woven polypropylene ply. Polypropylene (PP) is very skin-friendly. The further layers of the patient electric blanket are thus enclosed by a non-woven polypropylene fabric. The first textile comfort layer and/or the second textile comfort layer are preferably air and/or water-tight. The non-woven polypropylene fabric ply preferably has a mass per unit area in a range of 10 g/m² to 40 g/m², for example a mass per unit weight of about 20 g/m², and is thus very light and cozy. The non-woven polypropylene fabric ply can be a thermally stabilizing spun-bonded non-woven fabric and can be sealed from the inside with a PE film, for example with a 12 g/m² light-weight PE film. This PE film is also used for thermally bonding the edges of the top side and bottom side.

Preferred embodiments of the invention are explained and described in greater detail in the following, referencing the enclosed drawings.

### BRIEF DESCRIPTION OF THE FIGURES

FIG. 1 illustrates one exemplary embodiment of the inventive patient electric blanket while a patient is being provided thermal treatment;

FIG. 2 illustrates a further exemplary embodiment of the inventive patient electric blanket in a schematic exploded view;

FIG. 3 is a schematic depiction of a sub-region of an inventive patient electric blanket;

FIG. 4 is a schematic depiction of the connection region of an inventive patient electric blanket; and,

FIG. 5 illustrates a further exemplary embodiment of the inventive patient electric blanket in a schematic exploded view.

### DETAILED DESCRIPTION

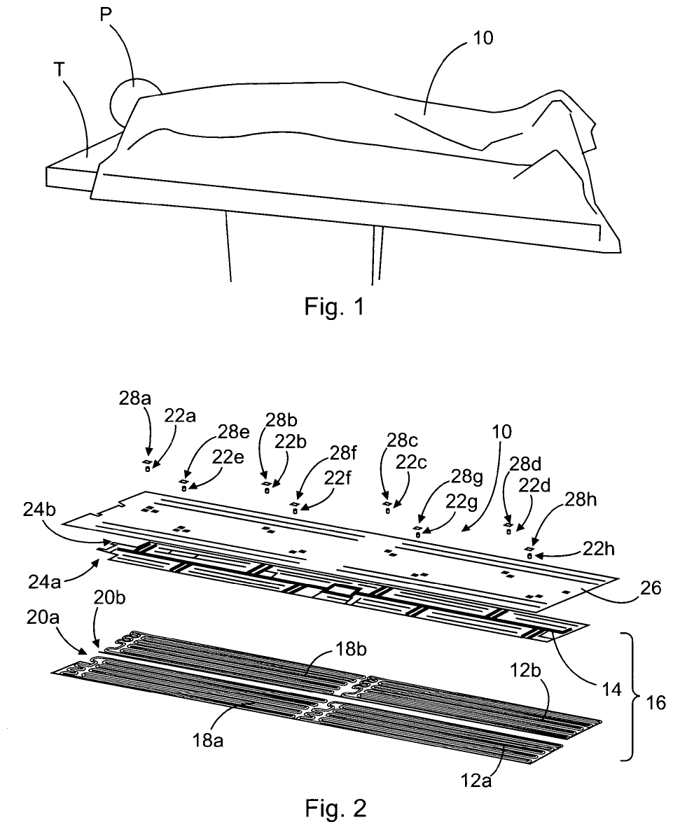

FIG. 1 illustrates a patient P lying on a table T. All of the patient P, with the exception of his head region, is covered with a patient electric blanket 10. The patient electric blanket 10 is electrically operable and provides heat to the patient P lying on the table T. The patient electric blanket 10 has a textile layer so that the shape of the patient electric blanket 10 adapts optimally to the body shape of the patient P.

FIG. 2 illustrates the structure of a multilayer electrically operable patient electric blanket 10. The patient electric blanket 10 includes two heating films 12a, 12b arranged adjacent to one another longitudinally, wherein each of the heating films 12a, 12b includes a heating track running in a meandering pattern. The heating track of the heating film 12a forms a first heating field 18a, wherein the heating track of the heating film 12b forms a second heating field 18b.

A contacting film 14 is arranged on top of the heating films 12a, 12b. The contacting film 14 contacts the temperature sensors 22a-22h. The contacting film 14 and the heating films 12a, 12b overlap in segments and together form a heating layer 16. The heating layer 16 thus includes the electrically conductive heating tracks of the heating films 12a, 12b and the contacting tracks of the contacting film 14, which contact the temperature sensors 22a-22h.

The heating tracks of the heating layer 16 are connected to power supply connectors 20a, 20b via which the heating tracks can be supplied with electrical energy from an energy source external to the blanket. The temperature sensors 22a-22h are connected via the contacting tracks of the contacting film 14 to the power supply connectors 24a, 24b, to which an external evaluation device can be attached for detecting the temperature.

The patient electric blanket 10 furthermore includes a textile base layer 26 connected to the heating layer 16 in a planar manner.

The textile base layer 26 is made of a non-woven fabric. The non-woven fabric provides thermal insulation and uniform temperature distribution across the entire patient electric blanket 10. Moreover, the non-woven fabric is soft, flexible, quiet, noiseless, and sound absorbing and ensures that the shape of the patient electric blanket 10 adapts optimally to the body shape of a patient P. The textile base layer 26 is embodied from a non-woven polyethylene terephthalate (PET) fabric. The non-woven PET fabric comprises 90% polyester fibers and 10% Co-PET fusible fibers, which after needling stabilize the non-woven fabric using a thermal process. The mass per unit area of the non-woven fabric is, for example, about 100 g/m². The textile base layer 26 and the heating layer 26 are bonded to one another by means of a hot melt adhesive. The bonding is done in a planar manner, so that the side of the contacting film 14 facing the base layer 26 and the regions of the heating films 12a, 12b not covered by the contacting film 14 on the side facing the base layer 26 are bonded to the base layer 26 using the adhesive. By using a textile base layer 26 embodied from non-woven fabric, the bonding process can be carried out at comparatively low bonding or laminating temperatures. The adhesive absorption capacity of the textile base layer 26 also prevents the formation of adhesive folds and the creation of uneveness during the bonding process.

The temperature sensors 22a-22h are each arranged in a temperature detection region of the patient electric blanket 10. The base layer 26 has recesses in the temperature detection regions of the patient electric blanket 10, so that, following the bonding of heating layer 16 and base layer 26, the temperature sensors 22a, 22h can be placed onto the contacting film 14 of the heating layer 16 as surface-mounted sensors. The temperature sensors 22a-22h are embodied as thermistors, specifically negative temperature coefficient thermistors. The temperature sensors 22a-22h are covered with a textile cover material 28a-28h. The textile cover material 28a, 28h is formed by affixable cover patches that are glued onto the temperature sensors 22a-22h and the base layer 26 in the region of the temperature sensors 22a-22h. The cover material 28a-28h is embodied from the same non-woven material as the base layer 26.

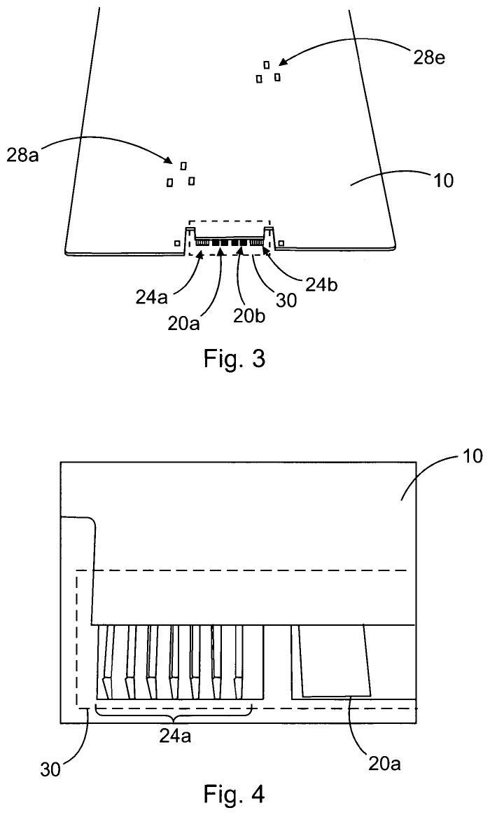

FIGS. 3 and 4 illustrate a connection region 30 of a patient electric blanket 10. The connection region 30 includes a plurality of power supply connectors 20a, 20b, via which heating tracks of the patient electric blanket 10 can be supplied with electrical energy.

Moreover, the connection region 30 includes a plurality of sensor connectors 24a, 24b that are connected to the temperature sensors 22a, 22h of the patient electric blanket 10 and to which an external evaluation device can be connected for detecting temperature.

The patient electric blanket 10 can be embodied as a single-use or disposable article, wherein a control module can be connected to the connection region 30 of the patient electric blanket 10. The control module preferably includes the energy source for providing electrical energy for the heating tracks and the evaluation device for detecting temperature. Thus the control module can regulate the temperature on the patient electric blanket 10 taking into account the temperature values measured by the temperature sensors 22a-22h. The control module is preferably designed for multiple usages and can thus be connected to different electric blankets 10 for patients for operating said patient electric blankets 10.

The power supply connectors 20a, 20b are components of the heating films 12a, 12b. The sensor connectors 24a, 24b are components of the contacting film 14. The heating layer 16 thus includes two heating films 12a, 12b and one contacting film 14.

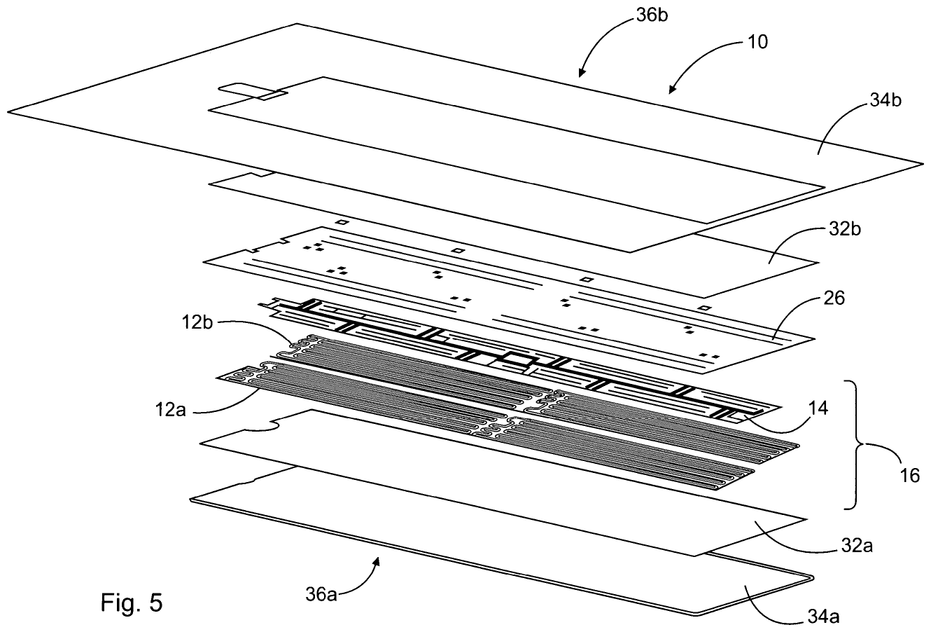

FIG. 5 illustrates an electrically operable patient electric blanket 10 having two heating films 12a, 12b arranged longitudinally adjacent to one another, wherein each of the heating films 12a, 12b includes a heating track with a meandering pattern. A contacting film 14 is arranged on top of the heating films 12a, 12b. The contacting film 14 and the heating films 12a, 12b overlap one another in segments and together form one heating layer 16.

The patient electric blanket 10 furthermore include a textile base layer 26 connected to the heating layer 16 in a planar manner. The textile base layer 26 is embodied from a non-woven polyethylene terephthalate fabric.

The patient electric blanket 10 furthermore includes two textile thermal insulation layers 32a, 32b. The first textile thermal insulation layer 32a is arranged on the side of the heating layer 16 facing away from the base layer 26 and is connected to the heating layer 16 in a planar manner. The second textile thermal insulation layer 32b is arranged the side of the base layer 26 facing away from the heating layer 16 and is connected to the base layer 26 in a planar manner. The first textile thermal insulation layer 32a and the second textile thermal insulation layer 32b each include two non-woven polyethylene terephthalate plies.

A first textile comfort layer 34a is arranged on the outside of the first textile thermal insulation layer 32a and is connected to the first textile thermal insulation layer 32a in a planar manner. The first textile comfort layer 34a includes an outside patient contact surface 36a, which forms a first exterior side of the patient electric blanket 10. A second textile comfort layer 34b is arranged on the outside of the second textile thermal insulation layer 32b and is connected to the second textile thermal insulation layer 32b in a planar manner. The second textile comfort layer 34b includes an outside patient contact surface 36b, which forms a second exterior side of the patient electric blanket 10. The first textile comfort layer 34a and the second textile comfort layer 34b are each embodied as a non-woven polypropylene fabric ply.

### REFERENCE NUMBERS

10 Patient electric blanket
12a, 12b Heating films
14 Contacting film
16 Heating layer
18a, 18b Heating fields
20a, 20b Power supply connectors
22a-22h Temperature sensors
24a, 24b Sensor connectors
26 Base layer
28a-28h Cover material
30 Connection region
32a, 32b Thermal insulation layers
34a, 34b Comfort layer
36a, 36b Patient contact surfaces
P Patient
T Table

1. A multi-layer electrically operable patient electric blanket comprising,
a heating layer that has one or a plurality of electrically conductive and non-destructively deformable heating tracks; and
a textile base layer connected to the heating layer in a planar manner.

2. The multi-layer electrically operable patient electric blanket according to claim 1, wherein
the textile base layer is embodied from a non-woven fabric or a polyethylene terephthalate non-woven fabric.

3. The multi-layer electrically operable patient electric blanket according to claim 1, wherein
the textile base layer is arranged immediately on top of the heating layer or on one or a plurality of heating tracks of the heating layer.

4. The multi-layer electrically operable patient electric blanket according to claim 1, wherein
the textile base layer and the heating layer are bonded to one another.

5. The multi-layer electrically operable patient electric blanket according to claim 1, wherein the multi-layer electrically operable patient electric blanket comprises
one or a plurality of temperature sensors, wherein the one or the plurality of temperature sensors are each arranged in a temperature detection region of the multi-layer electrically operable patient electric blanket.

6. The multi-layer electrically operable patient electric blanket according to claim 5, wherein
the one or the plurality of temperature sensors are each covered by a cover material or a textile cover material.

7. The multi-layer electrically operable patient electric blanket according to claim 1, wherein the multi-layer electrically operable patient electric blanket comprises a connection region with:
one or a plurality of electrical power supply connectors, via which electrical energy from an energy source external to the multi-layer electrically operable patient electric blanket can be provided to the one or the plurality of heating tracks; and/or
one or a plurality of sensor connectors that are connected to the one or the plurality of temperature sensors and to which an external evaluation device for temperature detection can be connected.

8. The multi-layer electrically operable patient electric blanket according to claim 7, wherein
the heating layer includes one or a plurality of heating films carrying heating tracks.

9. The multi-layer electrically operable patient electric blanket according to claim 8, wherein
one or a plurality of electrical power supply connectors are components of the one of the plurality of heating films.

10. The multi-layer electrically operable patient electric blanket according to claim 7, wherein
the one or the plurality of sensor connectors are components of a contacting film, wherein the contacting film includes one or a plurality of contacting tracks connected to the one or the plurality of temperatures sensors.

11. The multi-layer electrically operable patient electric blanket according to claim 10, wherein
the one or the plurality of heating tracks run in a meandering pattern within a heating region, wherein the heating region extends across at least 70% of the patient electric blanket or across at least 80% thereof.

12. The multi-layer electrically operable patient electric blanket according to claim 1, wherein the multi-layer electrically operable patient electric blanket comprises:
a first textile thermal insulation layer that is connected in a planar manner to the heating layer on a side of the heating layer facing away from the base layer; and/or
a second textile thermal insulation layer that is connected in a planar manner to the base layer on the side of the base layer facing away from the heating layer.

13. The multi-layer electrically operable patient electric blanket according to claim 12, wherein
the first textile thermal insulation layer and/or the second textile thermal insulation layer each include one or a plurality of non-woven fabric plies or two non-woven fabric plies or polyethylene terephthalate non-woven fabric plies.

14. The multi-layer electrically operable patient electric blanket according to claim 1, wherein the multi-layer electrically operable patient electric blanket comprises:
a first textile comfort layer that forms a first outer side of the patient electric blanket; and/or
a second textile comfort layer that forms a second outer side of the patient electric blanket.

15. The multi-layer electrically operable patient electric blanket according to claim 14, wherein
the first textile comfort layer and/or the second textile comfort layer each include at least one non-woven fabric ply or a non-woven polypropylene ply.
